(19) United States
(12) Patent Application Publication
(10) Pub. No.: US 2005/0009776 A1
(43) Pub. Date: Jan. 13, 2005

(54) Method of treating atrial fibrillation or atrial flutter

(75) Inventors: Shrikant Gadgil, Deerfield, IL (US); William Wheeler, Cary, NC (US); Shawn McDonald, Gurnee, IL (US); Stephen O'Dell, Midlothian, VA (US)

Correspondence Address: STERNE, KESSLER, GOLDSTEIN & FOX PLLC, 1100 NEW YORK AVENUE, N.W., WASHINGTON, DC, 20005, US

(73) Assignees: Aderis Pharmaceuticals, Inc., Richmond, VA (US); Fujisawa Healthcare, Inc., Deerfield, IL (US)

(21) Appl. No.: 10/831,341

(22) Filed: Apr. 26, 2004

Related U.S. Application Data
(60) Provisional application No. 60/464,957, filed on Apr. 24, 2003.
(60) Provisional application No. 60/514,009, filed on Oct. 27, 2003.

(51) Int. Cl. A61K 31/7076

(52) U.S. Cl. 514/46

(57) ABSTRACT

The present invention relates to the use of N⁶-cyclopentyl-5′-(N-ethyl)carboxamidoadenosine (DTI-0009) or a pharmaceutically acceptable salt or ester thereof in the treatment of atrial fibrillation or atrial flutter in a human. Especially an acute attack of atrial fibrillation or atrial flutter is treated by the method of this invention.

### BACKGROUND OF THE INVENTION

This application claims priority benefit under 35 U.S.C. § 119(e) of U.S. Provisional Application No. 60/464,957, filed Apr. 24, 2003, and U.S. Provisional Application No. 60/514,009, filed Oct. 27, 2003. The entirety of each of these applications is incorporated by reference herein.

### FIELD OF THE INVENTION

The present invention relates to the use of N⁶-cyclopentyl-5′-(N-ethyl)carboxamidoadenosine (DTI-0009) or a pharmaceutically acceptable salt or ester thereof in the treatment of atrial fibrillation or atrial flutter in a human.

### RELATED ART

In the mammalian heart, in order to circulate blood, contraction of the ventricles is initiated by an electrical impulse in the sinoatrial (SA) node that then passes through the atrioventricular (AV) node to the ventricles. Consistent rhythmic impulses produce a regular heart beat when the heart is functioning normally.

Various dysfunctions can occur in the heart to disturb the regular heart beat pattern and produce arrhythmias. Atrial fibrillation is the most common, sustained serious cardiac arrhythmia and is estimated to affect 2.2 million patients in the United States and approximately 6 million people worldwide. It is characterized by irregular wave fronts of atrial activation at rates of 350 to 600 beats per minute (bpm). The pumping effectiveness of the heart is depressed in both atrial fibrillation and atrial flutter, resulting in extensive functional and structural changes. Approaches to the management of atrial fibrillation and flutter are reduction of the ventricular response rate (VRR), restoration of sinus rhythm, and prevention of thromboembolic events. However, all clinically available drugs, except digoxin, that slow VRR during atrial fibrillation have the major limitation of lowering blood pressure or decreasing ventricular systolic function, which can limit clinical effectiveness. Even adenosine-based treatment can trigger side-effects because adenosine is not able to distinguish between the four types of adenosine receptors. The adenosine A₁ receptor is involved in atrial fibrillation and atrial flutter.

Adenosine, acting through A₁ receptors, slows conduction through the atrioventricular (AV) node. However, adenosine has a 10 second half-life which precludes its use for the treatment of VRR in atrial fibrillation. Adenosine is also a non-selective receptor agonist that can activate all four subtypes of adenosine receptors (A₁, A_{2A}, A_{2B}, and A₃). Thus, adenosine can cause decreases in systemic blood pressure through its activation of the A_{2A} and A_{2B} receptors on blood vessels. Therefore, an agent that selectively activates the A₁ adenosine receptor controlling AV nodal conduction without altering blood pressure would have a significant advantage over adenosine itself for the treatment of supraventricular tachyarrhythmias.

N⁶-cyclopentyl-5′-(N-ethyl)carboxamidoadenosine (DTI-0009) is a selective adenosine A₁ receptor agonist. International Patent Publication WO 01/37845 describes that N₆-cyclopentyl-5′-(N-ethyl)carboxamidoadenosine possesses particularly desirable pharmacological properties useful in the treatment of heart rhythm disturbances. Such heart rhythm disturbances include supraventricular tachycardia (PSVT) and atrial fibrillation/flutter (AF). WO 01/37845 describes parenteral, subcutaneous, intravenous, intramuscular, intraperitoneal, transdermal, buccal, or ocular routes of administration. WO 01/37845 describes suitable dosages of DTI-0009 to be administered by intravenous infusion, such as about 0.001 μg/kg to about 100 μg/kg, preferably about 0.005 μg/kg to about 1 μg/kg, more preferably about 0.01 μg/kg to about 0.5 μg/kg, to treat a heart rhythm disturbance.

The selectivity of DTI-0009 for adenosine A₁ receptors provides for more effective dosing and fewer side-effects. However, a need in the art exists for a clinically effective dosing regime for treating acute atrial fibrillation or atrial flutter that reduces the heart rate, atrioventricular (AV) node conduction, and atrial contractility with minimal side-effects, such as blood pressure changes.

### SUMMARY OF THE INVENTION

It has been found that a safe and a clinically effective method of treating a patient suffering from an attack of atrial fibrillation or atrial flutter involves a dosing regime that includes administering first a loading dose (LD) of N⁶-cyclopentyl-5′-(N-ethyl)carboxamidoadenosine (DTI-0009) or a pharmaceutically acceptable salt or ester thereof by intravenous infusion to reduce the heart rate, preferably below 100 bpm, and thereafter, if necessary, administering a maintenance dose of DTI-0009 to keep the heart rate down. It has also been found that the dosage to effectively treat an acute attack of atrial fibrillation or atrial flutter without deleterious side-effects is higher than the preferred dosages suggested in WO 01/37845.

Accordingly, the present invention provides a method for the treatment of atrial fibrillation or atrial flutter in a human, comprising administering intravenously a loading dose (LD) of an active agent which is N⁶-cyclopentyl-5′-(N-ethyl)carboxaamidoadenosine or a pharmaceutically acceptable salt or ester thereof to a human in need of said treatment in an amount of from about 0.5 μg/kg, preferably from about 1.25 μg/kg, to less than about 25.0 μg/kg and, optionally, thereafter administering a maintenance dose (MD) of said active agent as an intravenous infusion for a time period necessary. Suitably, the LD is administered over about 30 seconds to about 1 hour. Another suitable period of time for administering the LD is from about 1 minute to about 15 minutes.

Preferably, the LD is administered in an amount of from about 1.25 μg/kg to about 12.0 μg/kg. Accordingly, the present invention provides a method for the treatment of atrial fibrillation or atrial flutter in a human, comprising administering intravenously a loading dose of an active agent which is N⁶-cyclopentyl-5′-(N-ethyl)carboxamidoadenosine or a pharmaceutically acceptable salt or ester thereof to a human in need of said treatment in an amount of from about 1.25 μg/kg to about 12.0 μg/kg and, optionally, thereafter administering a maintenance dose (MD) of said active agent as an intravenous infusion for a time period necessary. Another suitable range for the LD is from about 2.5 μg/kg to about 10.0 μg/kg, suitably 4.0 μg/kg to about 10.0 μg/kg. Advantageous dosages for the LD include about 4.0 μg/kg, about 5.0 μg/kg, about 7.5 μg/kg, and about 10.0 μg/kg. The LD can be administered by continuous infusion or as a bolus injection. Suitably, the MD is administered at a rate of from about about 0.01 μg/kg/min to about 5.0 μg/kg/min, more suitably from about 0.01 μg/kg/min to about 1.0 μg/kg/min. Another suitable range for the rate of administering the MD is 0.5 μg/kg/min to about 5 μg/kg/min. Suitable rates for administering the MD also include about 1.75 μg/kg/hr, about 2.25 μg/kg/hr, and 2.75 μg/kg/hr. The maintenance dose is suitably administered within up to about 72 hours, more suitably from about 1 hour to about 72 hours, preferably up to 24 hours, more preferably up to 20 hours.

The present invention also provides a method for the treatment of atrial fibrillation or atrial flutter in a human, comprising administering a dose of N⁵-cyclopentyl-5′-(N-ethyl)carboxamidoadenosine or a pharmaceutically acceptable salt or ester thereof to a human in need of said treatment by intravenous infusion of about 2.0 μg/kg to about 12.0 μg/kg, preferably about 4.0 μg/kg to about 10.0 μg/kg. The dose is suitably administered within from about 1 minute to about 30 minutes, and preferably within 15 minutes.

Further, the present invention provides a dosing regime for treating atrial fibrillation or atrial flutter in a human, comprising an intravenous loading dose of an active agent which is N⁶-cyclopentyl-5′-(N-ethyl)carboxamidoadenosine or a pharmaceutically acceptable salt or ester thereof of from about 0.5 μg/kg, preferably from about 1.25 μg/kg, to less than about 25.0 μg/kg, wherein said loading dose is administered to said human within from about 6 seconds to about 60 minutes, followed by an optional maintenance dose of said active agent as an intravenous infusion at a rate of from about 0.01 μg/kg/min to about 5 μg/kg/min, preferably from about 0.5 μg/kg/min to about 5.0 μg/kg/min, more preferably from about 0.01 μg/kg/min to about 1.0 μg/kg/min. Advantageously, the loading dose is administered within from about 30 seconds to about 30 minutes, preferably within from about 1 minute to 15 minutes. The maintenance dose is suitably administered up to about 72 hours, more suitably from about 1 hour to about 72 hours, preferably up to 24 hours, more preferably up to 20 hours. A suitable range for the LD is from about 2.5 μg/kg to about 10.0 μg/kg, suitably 4.0 μg/kg to about 10.0 μg/kg. This dosing regime is especially suitable for treating an acute attack of atrial fibrillation or atrial flutter.

The present invention also provides a method of achieving a therapeutic plasma concentration of an active agent which is N⁶-cyclopentyl-5′-(N-ethyl)carboxamidoadenosine or a pharmaceutically acceptable salt or ester thereof for treating atrial fibrillation or atrial flutter in a human in need of such treatment, comprising administering intravenously a loading dose of said active agent of from about 0.5 μg/kg, preferably from about 1.25 μg/kg, to about 12.0 μg/kg, wherein said loading dose is administered to said human within from about 6 seconds to about 60 minutes, preferably from about 30 seconds to about 60 minutes, followed by an optional maintenance dose of said active agent as an intravenous infusion at a rate of from about 0.01 μg/kg/min to about 5 μg/kg/min, suitably 0.01 μg/kg/min to about 1.0 μg/kg/min. Advantageously, the MD is within the range of about 0.025 μg/kg/min and 0.1 μg/kg/min. Another advantageous range for the rate of administering the MD is from about 0.5 μg/kg/min to about 5.0 μg/kg/min.

Additional embodiments and advantages of the invention will be set forth in part in the description that follows, and in part will be obvious from the description, or may be learned by practice of the invention. The embodiments and advantages of the invention will be realized and attained by means of the elements and combinations particularly pointed out in the appended claims.

It is to be understood that both the foregoing general description and the following detailed description are exemplary and explanatory only and are not restrictive of the invention, as claimed.

### BRIEF DESCRIPTION OF THE FIGURES

FIG. 1 depicts the effect of DTI-0009 treatment on ventricular response rate (VRR) (bpm) during sinus rhythm (solid triangles), fixed atrial pacing at 250 msec (open squares), and fixed atrial pacing at 400 msec (solid diamonds). All values are treatment effect (estimated mean difference between the change from baseline for active dose and the change from baseline for normal saline control from an ANCOVA model). A: during treatment; B: 10 minutes post treatment. Statistically significant treatment effect: *p<0.05; **p<0.01, ***p=0.001.

FIG. 2 depicts the effect of DTI-0009 on change from baseline in AV WBCL (msec) during the treatment period (solid diamonds) and at 10 minutes post-treatment (open squares). All values are treatment effect (estimated mean difference between the change from baseline for active dose and the change from baseline for normal saline control from an ANCOVA model). Statistically significant treatment effect: *p<0.05; ***p=0.001.

### DETAILED DESCRIPTION OF THE INVENTION

N⁶-cyclopentyl-5′-(N-ethyl)carboxamidoadenosine (hereinafter also “DTI-0009”) has the following chemical formula:

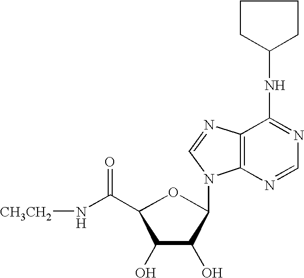

This compound is also known as selodenoson, 1-[6-(cyclopentylamino)-9H-purin-9-yl]-1-deoxy-N-ethyl-β-D-ribofuranuron-amide, N-5′-ethyl-N-6-(cyclopentyl)adenosine-5′-uronamide, or N⁶-cyclopentyladenosine-5′-ethylcarboxamide.

It has been discovered that the clinically effective dose of N⁶-cyclopentyl-5′-(N-ethyl)carboxamidoadenosine or a pharmaceutically acceptable salt or ester thereof for treating an acute attack of atrial fibrillation or atrial flutter is surprisingly larger than the dosages suggested in the art. It has also been found that the dosing regime of the present invention is significantly more effective than those currently used in the therapy for atrial fibrillation or atrial flutter.

Example 1 shows that the dosing regime of the present invention having a loading dose (LD) 2.5 μg/kg, 5.0 μg/kg, or 7.5 μg/kg of DTI-0009 followed by an optional maintenance dose (MD) of DTI-0009 caused a significant reduction in ventricular rate in patients having atrial fibrillation in a dose-dependent manner without significant blood pressure changes. Furthermore, the MD of DTI-0009 of 1.75 μg/kg/hr, 2.25 μg/kg/hr, or 7.5 μg/kg/hr resulted in sustained ventricular rate control. The LD was administered intravenously within 15 minutes. Also the study described in Example 2 showed significant dose-dependent inhibitory effects on the AV node with a longer duration of effects than conventional adenosine 30 minute infusion doses of 4.0 μg/kg or higher.

Suitable pharmaceutically acceptable salts of DTI-0009 for use according to the present invention include the conventional non-toxic salts or the quaternary ammonium salts which are formed, e.g., from inorganic or organic acids or bases. Examples of such acid addition salts include acetate, adipate, alginate, aspartate, benzoate, benzenesulfonate, bisulfate, butyrate, citrate, camphorate, camphorsulfonate, cyclopentanepropionate, digluconate, dodecylsulfate, ethanesulfonate, fumarate, glucoheptanoate, glycerophosphate, hemisulfate, heptanoate, hexanoate, hydrochloride, hydrobromide, hydroiodide, 2-hydroxyethanesulfonate, lactate, maleate, methanesulfonate, 2-naphtalenesulfonate, nicotinate, nitrate, oxalate, pamoate, pectinate, persulfate, 3-phenylpropionate, picrate, pivalate, propionate, succinate, sulfate, tartrate, thiocyanate, tosylate, and undecanoate. Base salts include ammonium salts, alkali metal salts such as sodium and potassium salts, alkaline earth metal salts such as calcium and magnesium salts, salts with organic bases such as dicyclohexylamine salts, N-methyl-D-glucamine, and salts with amino acids such as arginine, lysine, and so forth. Also, the basic nitrogen-containing groups may be quaternized with such agents as lower alkyl halides, such as methyl, ethyl, propyl, and butyl chloride, bromides and iodides; dialkyl sulfates like dimethyl, diethyl, dibutyl, and diamyl sulfates, long chain halides such as decyl, lauryl, myristyl and stearyl chlorides, bromides and iodides, aralkyl halides like benzyl and phenethyl bromides and others.

Suitable pharmaceutically acceptable esters of DTI-0009 for use according to the present invention include organic acid esters of the hydroxyl groups at the 2′ and 3′ positions of the ribofuranose moiety. Ester groups are preferably of the type which are relatively readily hydrolyzed under physiological conditions. Useful esters include those having an acyl group selected from the group consisting of acetyl, propionyl, butyryl and benzoyl groups.

DTI-0009 is an adenosine A₁ receptor agonist with an affinity for the A₁ receptor that is 340 fold higher than for the A_{2A} receptor and 11 fold higher than for the A₃ receptor. DTI-0009 is three times more potent at slowing AV nodal conduction than at reducing heart rate. The effects of DTI-0009 are rate dependent, and DTI-0009 is more potent at slowing AV nodal conduction during periods of rapid atrial pacing than during normal sinus rhythm in the guinea pig. Therefore, DTI-0009 may be capable of terminating episodes of supraventricular arrythymias at doses that have little effect on normal heart rate or blood pressure.

The term “atrial fibrillation” refers to a condition where there is disorganized electrical conduction in the atria, resulting in ineffective pumping of blood into the ventricle.

The term “atrial flutter” refers to a rapid well organized contraction of the atrium at a rate of 250-350 contractions per minute. In atrial flutter, ventricular response rates are usually some multiple of 300 and ECG shows sawtooth waves.

DTI-0009 can be synthesized according to the methods described in U.S. Pat. Nos. 5,310,731 and 4,868,160. For example, DTI-0009 may be obtained from 2′,3′-O-isopropylideneinosine-5′-carboxylic acid by treatment with a suitable inorganic acid halide, such as thionyl chloride, to yield the intermediate, 6-chloro-2′,3′-O-isopropylidene-9-β-D-ribofuranosylpurine-5′-carbonyl chloride. The intermediate, 6-chloro-2′,3′-O-isopropylidene-9-β-D-ribofuiranosylpurine-5′-carbonyl chloride, (or the corresponding bromide, if, for example thionyl bromide is used instead of thionyl chloride) need not be isolated in a pure state.

The acid chloride moiety of 6-chloro-2′,3′-O-isopropylidene-9-O-D-ribofuranosylpurine-5′-carbonyl chloride (or the acid bromide of the corresponding bromo-analog) is significantly more readily displaced by nucleophilic reagents than the halide in the 6-position of the purine moiety. Therefore, 6-chloro-2′,3′-O-isopropylidene-9-β-D-ribofuranosylpurine-5′-carbonyl chloride is first reacted with ethylamine to yield an intermediate as a substituted carboxamide, wherein the halide is retained in the 6-position of the purine moiety.

The intermediate substituted carboxamide is subsequently reacted with cyclopentylamine, and the isopropylidene blocking group is removed with dilute acid to yield DTI-0009 having free hydroxyl groups in the 2′ and 3′ positions of the ribofuranose moiety. Instead of the isopropylidene blocking group, other acid-stable blocking groups can also be used to protect the 2′-hydroxyl and 3′-hydroxyl groups of the ribofuranose moiety during the step of treatment with the inorganic acid halide.

In the method of treating atrial fibrillation or atrial flutter according to the present invention, DTI-0009 or a pharmaceutically acceptable salt or ester thereof can be administrated intravenously as a loading dose (LD) of from about 0.5 μg/kg, preferably from about 1.25 μg/kg, to less than about 25.0 μg/kg. Optionally after the administration of the LD, a maintenance dose (MD) is administered by intravenous infusion for a time period necessary. Other suitable loading doses include from about 0.01 μg/kg to about 1,000 μg/kg, suitably from about 0.1 μg/kg to about 100 μg/kg, more suitably from about 0.5 μg/kg to about 50 μg/kg, from about 2.0 μg/kg to about 25.0 μg/kg, from about 2.5 μg/kg to about 25.0 μg/kg, more suitably from about 1.25 μg/kg to about 12.0 μg/kg, from about 4.0 μg/kg to about 10.0 μg/kg. Advantageous dosages for the LD include about 4.0 μg/kg, about 5.0 μg/kg, about 7.5 μg/kg, and about 10.0 μg/kg. Suitable amount for treatment also includes a loading dose of about 2.5 μg/kg. The intravenous administration of the loading dose may consist of a single bolus injection or a continuous infusion. The loading dose can be administered within from about 6 seconds to about 60 minutes, preferably within from about 30 seconds to about 30 minutes. A suitable period of time for administering the LD includes the period of from about 10 minutes to about 40 minutes, preferably from about 15 minutes to about 30 minutes. Advantageously, the LD is administered within from about 1 minute to about 15 minutes. Specifically the bolus injection can be administered within a period of from about 6 seconds to about 2 minutes, preferably from about 6 seconds to less than 1 minute, more preperably from about 6 seconds to about 30 seconds. Suitably, lower loading doses, for example doses of about 3 μg/kg or lower, are administered as bolus injections and higher loading doses are administered using longer continuous intravenous infusion.

In a method of the present invention, treatment of atrial fibrillation or atrial flutter in a human can comprise administering a dose of DTI-0009 or a pharmaceutically acceptable salt or ester thereof to a human in need of said treatment by intravenous infusion of about 2.0 μg/kg to about 12.0 μg/kg, preferably of about 4.0 μg/kg to about 10.0 μg/kg. Suitable amounts for treatment also include, but are not limited to, 2.0 μg/kg, 4.0 μg/kg, 6.0 μg/kg, 8.0 μg/kg, 10 μg/kg or 12 μg/kg of DTI-0009, or pharmaceutically acceptable salt or ester thereof. The dose of DTI-0009 can be administered within from about 1 minute to about 30 minutes, preferably within about 15 minutes.

In the method of the present invention, the MD can be administered at a rate of from about 0.01 μg/kg/min to about 50 μg/kg/min, suitably from about 0.01 μg/kg/min to about 10 μg/kg/min, from about 0.01 μg/kg/min to about 1.0 μg/kg/min, from about 0.025 μg/kg/min to about 0.1 μg/kg/min. Advantageously, the MD is administered at a rate of from about about 0.5 μg/kg/min to about 5 μg/kg/min. Suitable rates for administering the MD also include about 1.75 μg/kg/hr, about 2.25 μg/kg/hr, and 2.75 μg/kg/hr. The maintenance dose is suitably administered up to about 72 hours, suitably from about 1 hour to about 72 hours, suitably up to 24 hours, more suitably up to 20 hours.

The dosing regime according to the present invention for treating an acute attack of atrial fibrillation or atrial flutter in a human comprises an intravenous loading dose of an active agent which is N⁶-cyclopentyl-5′-(N-ethyl)carboxamidoadenosine or a pharmaceutically acceptable salt or ester thereof of from about 0.5 μg/kg, preferably from about 1.25 μg/kg, to less than about 25.0 μg/kg, and preferably from about 0.5 μg/kg, more preferably from about 1.25 μg/kg, to about 12.0 μg/kg, wherein said loading dose is administered to said human within from about 6 seconds to about 60 minutes, preferably from about 30 seconds to about 60 minutes, followed by an optional maintenance dose of said active agent as an intravenous infusion at a rate of from about 0.01 μg/kg/min to about 5.0 μg/kg/min, suitably from about 0.5 μg/kg/min to about 5 μg/kg/min, preferably from about 0.01 μg/kg/min to about 1.0 μg/kg/min. Advantageously, the loading dose is administered within from about 30 seconds to about 30 minutes, suitably within from about 1 minute to 15 minutes. The maintenance dose is suitably administered up to 72 hours, suitably from about 1 hour hour to about 72 hours, up to 24 hours, suitably up to 20 hours.

The method of achieving a therapeutic plasma concentration of DTI-0009 or a pharmaceutically acceptable salt or ester thereof for treating atrial fibrillation or atrial flutter in a human in need of such treatment, comprises administering intravenously a loading dose of the active agent of from about 0.5 μg/kg, preferably from about 1.25 μg/kg, to about 12.0 μg/kg, and more preferably from more than 2.5 μg/kg to about 10.0 μg/kg, wherein said loading dose is administered to said human within from about 6 seconds to about 60 minutes, followed by an optional maintenance dose of the active agent as an intravenous infusion at a rate of from about 0.5 μg/kg/min to about 5 μg/kg/min.

The intravenous pharmaceutical preparations of the present invention are manufactured in a manner that is itself known, for example, by means of conventional mixing, dissolving, or lyophilizing processes. Thus, suitable pharmaceutical preparations for use in the present invention include aqueous solutions of DTI-0009 or a pharmaceutically acceptable salt or ester thereof in water-soluble form, for example, water-soluble salts and alkaline solutions. In addition, suspensions of the active compounds as appropriate oily injection suspensions may be administered. Suitable lipophilic solvents or vehicles include fatty oils, for example, sesame oil, or synthetic fatty acid esters, for example, ethyl oleate or triglycerides or polyethylene glycol-400 (the compounds are soluble in PEG-400). Aqueous injection suspensions may contain substances that increase the viscosity of the suspension, and include, for example, sodium carboxymethyl cellulose, sorbitol, and/or dextran. Optionally, the suspension may also contain stabilizers.

The intravenous pharmaceutical preparations can, if desired, also contain other compatible therapeutic agents. Examples of useful therapeutic agents that can be co-administered with DTI-0009 or a pharmaceutically acceptable salt or ester thereof include verapamil, quinidine, procainamide, diisopyramide, flecainide, ibutilide, dofetilide, amiodarone, sotalol, diltiazem, esmolol, propranolol, metoprolol, and digoxin. It can be useful to administer simultaneously, before or after, an anticoagulating agent to the patient in combination with DTI-0009 or a pharmaceutically acceptable salt or ester thereof.

The following examples are illustrative, but not limiting, of the method and compositions of the present invention. Other suitable modifications and adaptations of the variety of conditions and parameters normally encountered in clinical therapy and which are obvious to those skilled in the art are within the spirit and scope of the invention.

### EXAMPLE 1

### Ventricular Rate (VR) Control in Atrial Fibrillation with DTI-0009

A randomized, parallel-group, open label, dose-ranging study of DTI-0009 as a 15-minute intravenous loading dose (LD) followed by a maintenance dose (MD) infusion for up to 20 hours in adult patients with rapid atrial fibrillation (AF) (>120 bpm) was conducted. 78 patients were randomized to receive one of three LD regimens. If the ventricular rate (VR) was between 70 and 90 bpm 5 minutes after the end of the LD, then patients were randomized to one of the following three MDs of DTI-0009: 1.75 μg/kg/hr, 2.25 μg/kg/hr, or 2.75 μg/kg/hr. The MD was adjusted to keep the VR within the 70-90 bpm range.

63 of 69 patients completed the LD, 20 patients received a MD, and 18 patients completed at least 3 hours of MD. The VR remained below 100 bpm across all the dosing regimens during the 3-hour MD. The results are shown in Table 1 below.

TABLE 1

| | |
|---|---|
| LD | Time/min |

| (μg/kg) | Baseline | 5 | 10 | 15 | 20 |
|---|---|---|---|---|---|
| 2.5 | 143 | 129^{a} | 117^{a} | 113^{b} | 115^{b} |
| 5.0 | 138 | 115^{b} | 104^{b} | 108^{b} | 111^{b} |
| 7.5 | 143 | 110^{b,c} | 99^{b,c} | 105^{b} | 106^{b} |

^{a}p < 0.05 compared to Baseline
^{b}p < 0.001 compared to Baseline
^{c}p < 0.05 compared to the 2.5 μg/kg group

During the LD, systolic and diastolic blood pressures did not change significantly. The results indicate that in patients with atrial fibrillation, DTI-0009 administered as a 15-minute loading dose caused a significant decrease from baseline in ventricular rate that was dose-dependent without significant blood pressure changes. Furthermore, a maintenance dose of DTI-0009 resulted in sustained ventricular rate control.

### EXAMPLE 2

### Electrophysiologic Effects of DTI-0009 on Atrioventricular Nodal Function

Patient Population

An open-label, sequential group, dose-escalation study was conducted in electrophysiology laboratories at 11 sites in the United States between October 2000 and August 2001. The study protocol was approved by the institutional review board at each site, and patients provided assent and written informed consent prior to enrollment. Male and female patients (18 to 80 years of age), scheduled to undergo an electrophysiological (EP) study for a known or suspected cardiac arrhythmia or a catheter ablation procedure to an area other than the atrioventricular (AV) node, were enrolled in the study. Study procedures were performed after the completion of the scheduled EP procedure and patients were to be in sinus rhythm at study baseline. Exclusion criteria included: greater than first degree AV block, previous ablation of the AV node or AV node slow pathway, presence of an AV Wenckebach cycle length (AV WBCL)≧500 msec, treatment with atropine during the scheduled EP procedure, systolic blood pressure <90 or >180 mmHg, significant hemodynamic instability, ventricular response rate (VRR)<50 bpm, consumption of caffeine-containing food or beverage within the previous 12 hours, treatment with dipyridamole or diltiazem within the respective 5 half lives or theophylline, aminophylline, treatment with a therapeutic adenosine antagonist within the previous 24 hours, heart transplant or myocardial infarction within 30 days, known hypersensitivity to adenosine, known human immunodeficiency virus infection, or clinically significant hematologic, neurologic, hepatic, or renal function impairment.

Treatments and Assessments

Each patient received 1 of 6 doses of DTI-0009 (0.25, 1.0, 2.5, 4.0, 10.0, or 25.0 μg/kg) diluted in normal saline or normal saline only (control). Study drug was administered as a 30-minute i.v. infusion at a rate of 1 mL/minute. Patients were enrolled in sequential dose groups, beginning with the lowest active dose group and continuing in increasing dose order; the final group received normal saline. The decision to proceed to the next highest dose group was based on the safety response in the previous dose group.

Electrophysiologic (EP), electrocardiogram (ECG), blood pressure measurements, and blood collection for clinical laboratory measurements were performed at baseline (−10 to 0 minutes), during treatment (between 20 and 30 minutes), and at 10 minutes post-treatment.

EP measurements included ventricular response rate (VRR) during fixed atrial pacing at 250 and 400 msec (VRR 250 and VRR 400), AV WBCL, the interval from atrium to His bundle (AH interval) during sinus rhythm and rapid fixed atrial pacing at 600 msec, AV node effective refractory period (AVNERP), and atrial effective refractory period (AERP). Ventricular response rate during sinus rhythm was derived from the sinus cycle length.

ECG measurements included PR interval, QRS complex duration, and QT interval. Efficacy was based on changes in EP and ECG parameters from baseline to the treatment period and to post treatment. Safety was based on the incidence of adverse events, blood pressure measurements, and clinical laboratory values.

Statistical Methods

Demographic and baseline information were summarized by dose group. Efficacy variables were summarized overall by dose group at baseline, during treatment, and at post-treatment; mean changes from baseline to treatment period and post-treatment were calculated. Treatment effects (the mean difference between change from baseline for DTI-0009 dose and change from baseline for normal saline) and confidence intervals (95% CI) were estimated from an analysis of covariance (ANCOVA) model with treatment as the main factor and baseline score as a covariate. Regression models were constructed to test for dose responses. Efficacy analyses included all patients who received at least 20 minutes of study drug infusion, had analyzable VRR 250 at baseline and during treatment, and were in sinus rhythm at baseline.

The incidence of adverse events, blood pressure measurements, and clinical laboratory results were summarized by dose group. Adverse events were coded using the medical dictionary for regulatory activities (MedDRA version 3.0). Safety analyses included all patients who received any duration of study drug infusion.

Results

Patient Disposition, Baseline Characteristics, and Study Drug Administration

A total of 73 patients were enrolled in the study, of which 63 received study drug or placebo and were included in the safety analysis; 60 patients completed the study and 56 were included in the efficacy analyses. None of the 5 patients enrolled in the DTI-0009 25.0 μg/kg group completed the study; 2 of these patients were withdrawn during their scheduled EP procedure (prior to study drug administration) and 3 patients were withdrawn due to adverse events during the treatment period (efficacy results were obtained for 2 of these patients).

The mean (±SD) age for all patients who received study drug was 53.7±17.4 years (range 19 to 80 years). The majority of patients were white (85.7%) and male (69.8%). Patient demographics were similar across all dose groups. The most common reasons for patients to be undergoing an EP study were ventricular tachycardia (30.2%), atrial fibrillation (19.0%), and atrial flutter (15.9%).

The median duration of study drug infusion for all patients was 30 minutes. Of the 63 patients who received study drug, 53 received DTI-0009 at doses of 0.25 to 25.0 μg/kg, and 10 patients received normal saline.

Electrophysiologic Effects

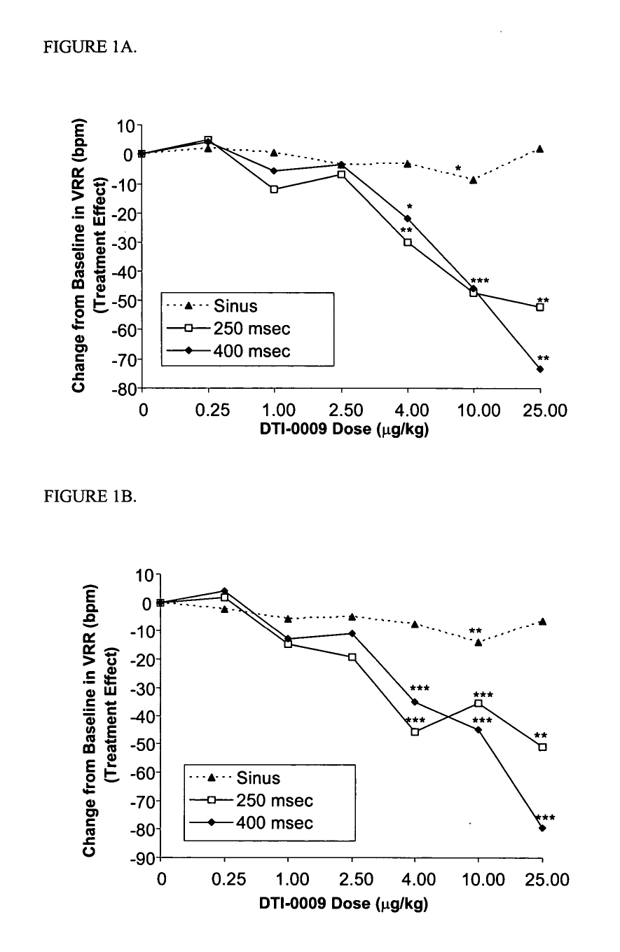

Mean baseline VRR 250 values for all dose groups were between 103.5 and 142.6 bpm. Statistically significant treatment effects (decreases from baseline compared with normal saline treatment) for VRR 250 were observed in the 4.0, 10.0, and 25.0 μg/kg DTI-0009 dose groups: 4.0 μg/kg: −29.7 bpm, CI −51.0, −8.4, p=0.008; 10.0 μg/kg: −47.4 bpm, CI −68.3, −26.4, p=0.001; 25.0 μg/kg: −52.1 bpm, CI: −85.7, −18.5, p=0.004. The results are shown in FIG. 1A. Similar treatment effects were observed for VRR 400 in the 4.0, 10.0, and 25.0 μg/kg DTI-0009 dose groups (p=0.024, p=0.001, and p=0.002, respectively). In addition, there were statistically significant dose responses across dose groups for both VRR 250 (p=0.002) and VRR 400 (p=0.001). Similar treatment effects and dose responses for VRR 250 and VRR 400 were observed at post-treatment. This is shown in FIG. 1B.

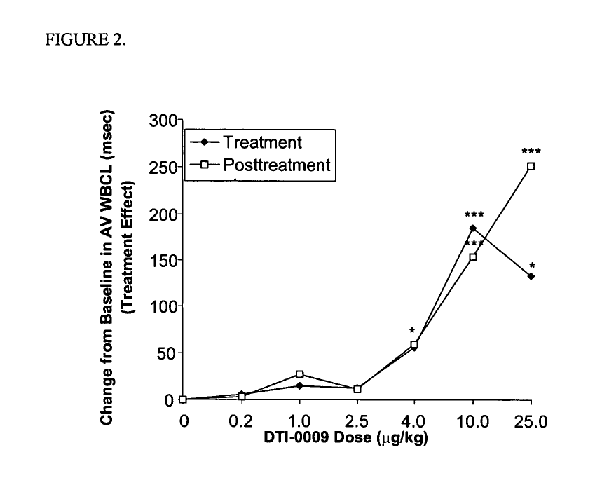

Mean baseline values for AV WBCL were between 335.0 and 382.2 msec for all dose groups. There were statistically significant treatment effects (increases from baseline) in AV WBCL during treatment in the 10.0 and 25.0 μg/kg DTI-0009 dose groups (p=0.001 and p=0.035, respectively), and at post-treatment in the 4.0, 10.0 and 25.0 μg/kg DTI-0009 dose groups (p=0.013, p=0.001, and p=0.001, respectively). These results are shown in FIG. 2. A statistically significant DTI-0009 dose response for AV WBCL was observed across all dose groups during treatment (p=0.001) and at post-treatment (p=0.001).

A statistically significant treatment effect (increase from baseline) for AH interval was observed for the treatment period during sinus rhythm in the 10.0 μg/kg DTI-0009 dose group (p=0.004) and during fixed rapid pacing at 600 msec in the 10.0 and 25.0 μg/kg DTI-0009 dose groups (p=0.001 and p=0.009, respectively) (see Table 2 below). In addition, there was a statistically significant dose response across all dose groups during sinus rhythm (p=0.005) and during fixed rapid pacing at 600 msec (p=0.001). There were similar treatment effects and dose responses for AH interval were also observed at post-treatment.

There was a statistically significant treatment effect (increase from baseline) for AVNERP during treatment in the 10.0 and 25.0 μg/kg DTI-0009 dose groups (p=0.001 and p=0.013, respectively) and at post-treatment in the 4.0, 10.0, and 25.0 μg/kg DTI-0009 dose groups (p=0.049, p=0.001 and p=0.003, respectively) (see Table 2 below). In addition, statistically significant dose responses for AVNERP were observed across dose groups during treatment (p=0.001) and at post-treatment (p=0.001).

There was a statistically significant reduction of AERP during treatment in the 10.0 μg/kg DTI-0009 dose group (p=0.035), and at post-treatment in the 1.0 and 10.0 μg/kg DTI-0009 dose groups (p=0.040 and p=0.012, respectively) (see Table 2).

TABLE 2
AH interval, AV Node Effective Refractory Period (AVNERP), and Atrial Effective
Refractory Period (AERP) During Treatment with DTI-0009.

| | AH interval (msec) | | | | | | | |
|---|---|---|---|---|---|---|---|---|
| DTI-0009 | Sinus Rhythm | | 600 msec Pacing | | AVNERP (msec) | | AERP (msec) | |
| Dose Group | Baseline | Treatment | Baseline | Treatment | Baseline | Treatment | Baseline | Treatment |
| (μg/kg) | (mean ± se) | Effect | (mean ± se) | Effect | (mean ± se) | Effect | (mean ± se) | Effect |
| Saline | 92.9 ± 5.6 | — | 113.7 ± 13.1 | — | 272.2 ± 18.8 | — | 231.1 ± 12.5 | — |
| 0.25 | 83.5 ± 6.2 | −2.8 | 90.4 ± 6.1 | 4.85 | 291.4 ± 14.4 | −7.03 | 242.0 ± 11.2 | −9.6 |
| 1.0 | 84.1 ± 7.8 | −0.1 | 109.4 ± 10.5 | 2.6 | 268.9 ± 22.8 | −1.0 | 223.3 ± 17.2 | −18.3 |
| 2.5 | 78.9 ± 10.2 | 7.1 | 94.3 ± 11.7 | 18.0 | 263.8 ± 24.2 | 16.0 | 221.3 ± 13.8 | −7.3 |
| 4.0 | 86.7 ± 6.3 | 7.2 | 109.6 ± 6.8 | 18.7 | 331.1 ± 36.2 | 82.4 | 223.3 ± 12.1 | 0.6 |
| 10.0 | 82.9 ± 4.9 | 21.1** | 106.5 ± 10.1 | 57.9*** | 248.8 ± 18.2 | 231.5*** | 228.8 ± 13.7 | −22.0* |
| 25.0 | 95.5 ± 4.5 | 14.2 | 146.0 ± 30.0 | 64.8** | 345.0 ± 5.0 | 198.3* | 240.0 ± 0.0 | 4.0 |

Treatment effect: The estimated mean difference between the change from baseline for active dose and the change from baseline for normal saline control from an ANCOVA model.
*p < 0.05;
**p < 0.01;
***p = 0.001.

There were no statistically significant changes in ECG parameters during the study, with the exception of paradoxic decreases from baseline in PR interval in the 1.0 μg/kg DTI-0009 dose group during treatment (treatment effect −42.2 msec, p=0.008), and at post-treatment (treatment effect −34.5 msec, p=0.023); and an increase in PR interval at post-treatment in the 10.0 μg/kg DTI-0009 dose group (treatment effect 42.9 msec, p=0.007).

Safety

A total of 29 patients (46.0%) experienced one or more adverse events during the study. The adverse events were consistent with the study population and pharmacological actions of DTI-0009. The most common events were atrial fibrillation (8 patients), chest pain or tightness (5 patients), and atrial flutter (4 patients). While the number of patients with any adverse event did not show a direct relationship to the dose of DTI-0009 given, adverse events were more commonly observed in the 25.0 μg/kg DTI-0009 dose group (see Table 3). A relationship to the DTI-0009 dose was observed for the incidence of atrial fibrillation, all cardiac adverse events, and all adverse events considered to be related to the study drug, with patients in the 25.0 μg/kg DTI-0009 dose group having the highest incidence of these events.

The majority of adverse events were mild or moderate in severity; only 3 patients (1 patient in the 10.0 μg/kg DTI-0009 group and 2 patients in the 25.0 μg/kg DTI-0009 group) had severe events (2 events of chest pain and 1 event of bradycardia). Three patients in the 25.0 μg/kg DTI-0009 dose group did not complete study drug infusion due to adverse events (chest pain, severe chest pain, and chest tightness and atrial fibrillation) considered to be related to study drug. No other patient discontinued from study drug treatment due to an adverse event.

TABLE 3
Incidence of Adverse Events.

| | Normal | DTI-0009 Dose Groups (μg/kg) | | | | | |
|---|---|---|---|---|---|---|---|
| | Saline | 0.25 | 1.0 | 2.5 | 4.0 | 10.0 | 25.0 |
| | (n = 10) | (n = 10) | (n = 10) | (n = 8) | (n = 10) | (n = 12) | (n = 3) |
| Any Adverse Event | 4 (40%) | 3 (30%) | 6 (60%) | 3 (38%) | 2 (20%) | 8 (67%) | 3 (100%) |
| Cardiac Disorders: | 1 (10%) | 2 (20%) | 1 (10%) | 1 (13%) | 2 (20%) | 6 (50%) | 2 (66.7%) |
| Atrial fibrillation | 0 | 1 (10%) | 0 | 1 (13%) | 2 (20%) | 3 (25%) | 1 (33%) |
| Atrial flutter | 1 (10%) | 1 (10%) | 1 (10%) | 0 | 0 | 1 (8%) | 0 |
| Atrial tachycardia | 0 | 0 | 0 | 0 | 0 | 0 | 1 (33%) |
| Bradycardia | 0 | 0 | 0 | 0 | 0 | 1 (8%) | 1 (33%) |
| Conduction disorders | 0 | 0 | 0 | 0 | 0 | 1 (8%) | 0 |
| General Disorders | 0 | 0 | 1 (10%) | 2 (25%) | 0 | 2 (17%) | 3 (100%) |
| Chest pain/tightness | 0 | 0 | 0 | 1 (13%) | 0 | 1 (8%) | 3 (100%) |
| Groin pain/swelling | 0 | 0 | 1 (10%) | 0 | 0 | 0 | 0 |
| Adverse Events | 0 | 0 | 1 (10%) | 0 | 2 (20%) | 7 (58%) | 3 (100%) |
| Related to Study Drug | | | | | | | |
| Cardiac Disorders (all) | 0 | 0 | 1 (10%) | 0 | 2 (20%) | 6 (50%) | 2 (66.7%) |
| General Disorders (all) | 0 | 0 | 0 | 0 | 0 | 1 (8%) | 3 (100%) |

There were no clinically significant changes in blood pressure (see Table 4) or clinical laboratory values for any patient during this study.

TABLE 4
Changes in Blood Pressure During Treatment with DTI-0009

| DTI-0009 | Systolic BP (mmHg) | | | Diastolic BP (mmHg) | | |
|---|---|---|---|---|---|---|
| Dose Group | | Change from Baseline | | | Change from Baseline | |
| (μg/kg) | Baseline | Treatment | Post-treatment | Baseline | Treatment | Post-treatment |
| Saline | 121.1 ± 15.9 | 4.2 ± 10.2 | 4.7 ± 16.0 | 72.6 ± 11.2 | 4.6 ± 7.4 | 7.6 ± 8.8 |
| 0.25 | 127.3 ± 16.4 | 8.8 ± 8.2 | 5.8 ± 9.5 | 72.9 ± 12.7 | 2.5 ± 8.1 | 5.2 ± 9.7 |
| 1.0 | 136.9 ± 27.6 | −0.1 ± 18.0 | 1.4 ± 21.5 | 71.5 ± 13.0 | 3.3 ± 9.3 | 1.0 ± 5.6 |
| 2.5 | 137.1 ± 17.2 | 1.0 ± 6.2 | 0.6 ± 9.0 | 81.7 ± 12.9 | 0.0 ± 8.4 | −1.6 ± 9.5 |
| 4.0 | 114.7 ± 15.0 | 3.8 ± 8.4 | 5.2 ± 10.2 | 67.8 ± 8.1 | 3.3 ± 8.4 | 0.6 ± 10.5 |
| 10.0 | 126.1 ± 16.5 | −3.2 ± 13.5 | −12.6 ± 20.4 | 76.0 ± 18.2 | −3.2 ± 11.2 | −7.2 ± 16.8 |
| 25.0 | 125.7 ± 7.1 | 23.0 ± 2.8 | 6.7 ± 8.1 | 72.0 ± 9.5 | 7.5 ± 2.1 | 1.0 ± 8.0 |

All values are reported as mean ± SD

In this study, the electrophysiologic effects of DTI-0009 administered as an infusion showed a dose-related inhibition of AV node conduction, as demonstrated by the reduction in VRR during fast atrial pacing (250 and 400 msec), and by the increases in AV WBCL and AH interval. Although this was not a randomized study, there were no statistically significant differences between dose groups for the baseline electrophysiologic measurements, and these measurements were similar to normal values reported by Taneja, T. et al. (PACE 24:16-21 (2001)).

Although there was no statistically significant treatment effect at lower DTI-0009 doses (0.25, 1.0, and 2.5 μg/kg), there was a statistically significant linear dose response to DTI-0009 at higher doses for the VRR 250 during the treatment period and at post-treatment. The magnitude of reduction in VRR 250 in the 4.0 and 10.0 μg/kg DTI-0009 groups may be clinically useful for VRR control in atrial flutter and atrial fibrillation. There was also a linear dose response for VRR 400 during treatment and at post-treatment with statistically significant treatment effects in the 4.0, 10.0, and 25.0 μg/kg DTI-0009 dose groups, although only one subject in 25.0 μg/kg group had VRR 400 measurements.

The reduction in VRR resulting from the DTI-0009 treatment during atrial pacing may be attributed to the prolongation of AV WBCL. The mean AV WBCL increased in the 4.0, 10.0, and 25 μg/kg DTI-0009 dose groups during treatment and at post-treatment, although the effect was not statistically significant in the 4.0 μg/kg group during treatment.

AH interval and AVNERP are additional electrophysiologic measurements used to assess the inhibition of AV node function. To break the AV node circuit during AV node re-entry, it is desirable to increase AVNERP more than AH interval, since this reduces the excitable gap (Talajic, M. et al., Circulation 86:870-877 (1992)). In this study, there were statistically significant increases in both AVNERP and AH interval in the 10.0 and 25.0 μg/kg DTI-0009 dose groups, and the increases were proportionately greater for AVNERP than for AH interval.

In general, the electrophysiologic effects of DTI-0009 were similar during the treatment period (after 20 to 30 minutes of study drug infusion) and at 10 minutes posttreatment, indicating that the effects were sustained at least up to 10 minutes and there was no hysteresis or lag effect.

The electrophysiologic effects of the DTI-0009 10.0 μg/kg infusion on AH interval during sinus rhythm and AV WBCL were similar to those reported elsewhere for a therapeutic dose of diltiazem (two diltiazem 25.0 mg i.v. boluses followed by a 10.0 mg i.v. infusion) used to control VRR during atrial fibrillation or atrial flutter (Talajic, M. et al., ibid.). The increases in AH interval during sinus rhythm and 600 msec atrial pacing were similar to those observed with another selective adenosine A₁ agonist (CVT 510) at a bolus dose of 10.0 μg/kg (Lerman, B. et al., J. Cardiovasc. Pharmacol. Therapeut. 6:237-245 (2001)).

The majority of adverse events were cardiac events such as atrial fibrillation and atrial flutter, which may not be clinically relevant in this patient population. There was no dose response relationship between doses of 0.25 to 10.0 μg/kg of DTI-0009 and the incidence of any individual adverse events, regardless of their relationship to study drug. Almost all adverse events in these dose groups were mild or moderate in severity, and no patients in these dose groups discontinued due to an adverse event. Thus, DTI-0009 appears to have an acceptable safety profile at doses of 0.25 to 10.0 μg/kg in this patient population.

There was a higher incidence of atrial fibrillation in the 10 μg/kg DTI-0009 dose group than in the lower dose groups, which may be attributable to the shortening of AERP at this dose. Chest pain is a common adverse event in patients receiving adenosine infusion (Cerqueria, M. D. et al., J. Am. Cardiol. 23:384-389 (1994)), and it is believed to be caused by the stimulation of cardiac afferent nerves, because it can occur in the absence of myocardial ischemia, and it is not experienced in heart transplant patients whose heart is denervated (Bertolet, B. D. et al., Circulation 93:1871-1876 (1996)). Therefore, chest pain associated with DTI-0009 may be non-ischemic. It has also been shown by Bertolet et al. (ibid.) that a selective A₁ antagonist (N-0861) can block chest discomfort and increases in AH interval in humans without significantly diminishing coronary flow during adenosine infusion. This indicates that the effect on the cardiac afferent nerves may be due to the Al receptor activation.

There were no statistically significant changes in QRS duration or QT interval during the study. There were decreases in PR interval in the 1.0 μg/kg DTI-0009 dose group during treatment and at post-treatment, and an increase in PR interval at post-treatment in the 10.0 μg/kg DTI-0009 dose group. No clear reason for the decrease in PR interval was identified, but the increase in PR interval can be attributed to prolongation of the AH interval.

Adenosine infusion is known to be associated with a decrease in blood pressure. In this study, there were no statistically significantly changes in blood pressure in any of the dose groups during treatment or at 10 minutes post-treatment. The absence of a blood pressure effect makes DTI-0009 potentially unique among available AV rate-blocking drugs.

This study demonstrated that DTI-0009 administered as a 30-minute i.v. infusion has significant inhibitory effects on the AV node at doses of at least 4.0 μg/kg and has an acceptable safety profile at doses up to 10.0 μg/kg.

### EXAMPLE 3

This study for determining the effects of DTI-0009 on atrioventricular (AV) node function enrolled 63 patients (19 women and 44 men; mean age 54) who were scheduled for a procedure other than AV node or slow pathway ablation. All patients had no greater than a first degree AV block and AV node Wenckebach Cycle Length of less than 500 msec. The patients had a 12-hour caffeine washout and no use of theophylline or aminophylline in the prior 24 hours. All patients were in sinus rhythm at the time of the study and all had a baseline heart rate of at least 50 bpm.

The study was a Phase I, multi-center, open-label, sequential dose escalation using DTI-0009 (0.25 μg/kg, 1.0 μg/kg, 2.5 μg/kg, 4.0 μg/kg, 10.0 μg/kg, and 25.0 μg/kg) or normal saline. Electrophysiologic studies (EPS) were performed at baseline, during infusion (20-30 min) and 10 minutes after discontinuation of DTI-0009 administration. DTI-0009 continued to have an effect on the above AV node variables at 10 minutes post-infusion suggesting longer biological effects than that of conventional adenosine. The results are shown in Table 5 below.

Normal saline and DTI-0009 at doses up to 2.5 μg/kg had negligible effects. Only three patients received 25.0 μg/kg that had a much greater effect on the AV node: two of these patients experienced transient, severe, non-ischemic chest pain that were reported as serious adverse events. A complete heart block occurred in one patient at the 10.0 μg/kg dose.

TABLE 5
Electrophysiologic Effects of DTI-0009.

| | Dose of DTI-0009 (μg/kg) | | |
|---|---|---|---|
| Change from | 4.0 | 10.0 | 25.0 |
| Baseline | (n = 9) | (n = 8) | (n = 2) |
| AH Interval | +9 msec (ns) | +24 msec | +14 msec (ns) |
| | | (p = 0.004) | |
| Wenkebach Cycle | +60 msec (ns) | +198 msec | +140 msec |
| Length | | (p = 0.001) | (p = 0.035) |
| AVNERP | +73 msec (ns) | +248 msec | +185 msec |
| | | (p = 0.001) | (p = 0.013) |
| AERP | +10 msec (ns) | −14 msec | +10 msec (ns) |
| | | (p = 0.035) | |
| HR at 250 msec | −49 bpm | −51 bpm | −45 bpm |
| pacing | (p = 0.008) | (p = 0.001) | (p = 0.004) |

This study showed significant dose-dependent inhibitory effects on the AV node with a longer duration of effects than conventional adenosine. DTI-0009 was well tolerated up to a dose of 10.0 μg/kg. Serious adverse events reported at 25.0 μg/kg were consistent with those of a selective A₁ agonist.

### EXAMPLE 4

A double blind, placebo-controlled study was designed to establish the i.v. dose range of DTI-0009 that safely reduces heart rates in patients with atrial fibrillation. Sixty-three patients with atrial fibrillation and baseline sustained heart rates between 110 and 200 bpm were randomized to receive placebo or DTI-0009 (2, 4, 6, 8, 10 or 12 μg/kg) in sequential dose groups as a 15-minute i.v. infusion. The patients' heart rates and blood pressures were measured periodically throughout the treatment period and for 75 and 80 minutes post-infusion, respectively. Safety assessments were made throughout the study and for up to three to seven days post-dose. The results are shown in Table 6.

Sixty-two of 63 patients completed the 15-minute infusion period. There were significant decreases in heart rates at all doses of DTI-0009 at both the 5 and 15-minute time points. There were no clinically significant changes in systolic or diastolic blood pressures in any treatment groups throughout the infusion period and for up to 80 minutes post-infusion.

TABLE 6
The effects of i.v. dosages of DTI-0009 on heart rate and
blood pressure

| | | | | 30 |
|---|---|---|---|---|
| | | | 15 minutes | minutes |
| | | 5 minutes after | after start of | after start |
| | Baseline | start of infusion | infusion | of infusion |
| Placebo (n = 14) | | | | |
| HR (bpm) | 132.1 | 132.7 | 125.3 | 122.1 |
| SBP (mmHg) | 126.7 | 122.6 | 118.4 | 122.4 |
| DBP (mmHg) | 70.1 | 69.0 | 74.9 | 69.1 |
| DTI-0009 | | | | |
| 2 μg/kg (n = 12) | | | | |
| HR (bpm) | 129.9 | 117.1^{a} | 102.4^{b} | 112.1^{a} |
| SBP (mmHg) | 130.7 | 136.6 | 134.4 | 129.5^{e} |
| DBP (mmHg) | 82.2 | 87.7 | 84.6 | 77.8^{e} |
| 4 μg/kg (n = 9) | | | | |
| HR (bpm) | 143.9 | 114.9^{a} | 100.1^{b} | 115.1^{a,d} |
| SBP (mmHg) | 132.1 | 127.4 | 132.8 | 122.0 |
| DBP (mmHg) | 75.0 | 76.3 | 68.7 | 72.2 |
| 6 μg/kg (n = 9) | | | | |
| HR (bpm) | 140.2 | 117.1^{a} | 100.4^{b} | 114.4^{a} |
| SBP (mmHg) | 122.1 | 126.8 | 126.8 | 115.4 |
| DBP (mmHg) | 69.4 | 72.9 | 70.4 | 68.7 |
| 8 μg/kg (n = 7) | | | | |
| HR (bpm) | 133.7 | 101.4^{a} | 90.9^{a} | 96.3^{a} |
| SBP (mmHg) | 131.6 | 141.4 | 145.3 | 134.4 |
| DBP (mmHg) | 82.3 | 85.1 | 80.3 | 77.4 |
| 10 μg/kg (n = 8) | | | | |
| HR (bpm) | 142.5 | 110.0^{b} | 95.4^{a} | 104.6^{a} |
| SBP (mmHg) | 128.3 | 140.6 | 125.8 | 126.1 |
| DBP (mmHg) | 75.9 | 83.1 | 73.0 | 71.3 |
| 12 μg/kg (n = 4) | | | | |
| HR (bpm) | 120.0 | 82.0^{a} | 74.5^{a} | 88.0^{a} |
| SBP (mmHg) | 119.5 | 131.5 | 122.5 | 118.5 |
| DBP (mmHg) | 80.5 | 82.5 | 78.5 | 68.5^{a} |

^{a}p < 0.05 compared to Baseline.
^{b}p < 0.001 compared to Baseline.
^{c}Blood pressure measurements were done 35 minutes after the start of the infusion.
^{d}There were only data from 8 patients available at this time point.
^{e}There were only data from 11 patients available at this time point.

This study demonstrated that i.v. doses of DTI-0009, ranging from 2 μg/kg to 12 μg/kg, significantly reduced heart rates compared to baseline without significantly altering blood pressures for patients with atrial fibrillation.

### EXAMPLE 5

A study was designed to determine the dose dependent pharmacokinetics (PK) of short i.v. infusions of DTI-0009 in healthy subjects. Ten healthy young volunteer (ages 21 to 35 years old) received a 30-minute infusion of 1 μg/kg DTI-0009, or 5 μg/kg DTI-0009 or saline on three different occasions. The subjects were hydrated by a saline infusion (200 ml/min) for one hour prior to and five hours after the start of the drug infusion. Serial plasma and urine samples were taken over 24 hours to measure DTI-0009 concentrations (as well as glucuronide, in urine only), creatinine, sodium and potassium by validated methods. The following renal function markers were assessed over time: urinary flow rate (UF), creatinine clearance (CL_{Crea}), sodium clearance (CL_{Na}) and potassium clearance (CL_{K}). Additionally, noncompartmental PK analysis was performed and instantaneous renal clearance (CLᵣₑₙ^{inst}) was estimated as a function of UF and CL_{crea}.

Initially, DTI-0009 transiently reduced diuresis, natriuresis and kaliuresis due to significant, dose-dependent inhibition of CL_{crea}, CL_{Na} and to a lesser extent CL_{K}. The inhibitory effects on CL_{Crea} were shorter (1-2 hours) than the effects on CL_{Na} (3-4 hours). Dose-depenent secondary effects (rebounds) in UF and natriuresis were apparent after 5-10 hours. About 42% of DTI-0009 was eliminated uncharged in urine and 3.7% as glucuronide. CLₜₒₜ and CLᵣₑₙ were reduced with increasing dose by 25% and 29%, respectiviely; CLₙₒₙᵣₑₙ appeared unchanged. CLᵣₑₙ^{inst} paralleled DTI-0009-induced transient reductions in UF and CL_{Crea}. Vdₛₛ approached body weight.

This study demonstrated that DTI-0009 transiently reduces glomerular filtration, presumably by renal vasoconstriction, as well as tubular reabsorption via activation of renal A₁-receptors. Counter-regulatory mechanisms seem to blunt these primary effects. This study also demonstrated that DTI-0009 is largely eliminated by renal tubular secretion, which is temporarily reduced by DTI-0009-induced renal A₁-receptor activation. This leads to dose- and time-dependent PK. DTI-0009 can be used in a method of reducing diuresis in a patient in need thereof.

Having now fully described this invention, it will be understood by those of ordinary skill in the art that the same can be performed within a wide and equivalent range of conditions, formulations and other parameters without affecting the scope of the invention or any embodiment thereof.

Other embodiments of the invention will be apparent to those skilled in the art from consideration of the specification and practice of the invention disclosed herein. It is intended that the specification and examples be considered as exemplary only, with a true scope and spirit of the invention being indicated by the following claims.

All patents and publications cited herein are fully incorporated by reference herein in their entirety.

1. A method for the treatment of atrial fibrillation or atrial flutter in a human, comprising administering intravenously a loading dose of an active agent which is N⁶-cyclopentyl-5′-(N-ethyl)carboxamidoadenosine or a pharmaceutically acceptable salt or ester thereof to a human in need of said treatment in an amount of from about 0.5 μg/kg to less than about 25.0 μg/kg and, optionally, thereafter administering a maintenance dose of said active agent as an intravenous infusion.

2. The method of claim 1, wherein said loading dose is administered in an amount of from about 1.25 μg/kg to less than about 25.0 μg/kg.

3. The method of claim 2, wherein said loading dose is administered in an amount of from about 1.25 μg/kg to about 12.0 μg/kg.

4. The method of claim 3, wherein said loading dose is administered in an amount of from about 2.0 μg/kg to about 12.0 μg/kg.

5. The method of claim 4, wherein said loading dose is administered in an amount of from about 4.0 μg/kg to about 10.0 μg/kg.

6. The method of claim 5, wherein said loading dose is about 4.0 μg/kg.

7. The method of claim 5, wherein said loading dose is about 5.0 μg/kg.

8. The method of claim 5, wherein said loading dose is about 7.5 μg/kg.

9. The method of claim 5, wherein said loading dose is about 10.0 μg/kg.

10. The method of claim 1, wherein said loading dose is administered within about 30 seconds to 1 hour.

11. The method of claim 10, wherein said loading dose is administered within about 30 minutes.

12. The method of claim 10, wherein said loading dose is administered within about 1 minute to about 15 minutes.

13. The method of claim 1, wherein said loading dose is administered by continuous infusion.

14. The method of claim 1, wherein said loading dose is administered as a bolus injection within a period of from about 6 seconds to about 2 minutes.

15. The method of claim 1, wherein said maintenance dose is administered at a rate of from about 0.01 μg/kg/min to about 1.0 μg/kg/min.

16. The method of claim 1, wherein said maintenance dose is administered of from about 0.5 μg/kg/min to about 5.0 μg/kg/min.

17. The method of claim 1, wherein said maintenance dose is administered over about 72 hours.

18. The method of claim 17, wherein said maintenance dose is administered over about 1 hour to about 72 hours.

19. The method of claim 17, wherein said maintenance dose is administered up to 24 hours.

20. The method of claim 19, wherein said maintenance dose is administered up to 20 hours.

21. The method of claim 1, wherein said maintenance dose is selected from the group consisting of about 1.75 μg/kg/hr, about 2.25 μg/kg/hr, and about 2.75 μg/kg/hr.

22. A method for the treatment of atrial fibrillation or atrial flutter in a human, comprising administering a dose of N⁶-cyclopentyl-5′-(N-ethyl)carboxamidoadenosine or a pharmaceutically acceptable salt or ester thereof to a human in need of said treatment by intravenous infusion of about 2.0 μg/kg to about 12.0 μg/kg.

23. The method of claim 22, wherein said dose is about 4.0 μg/kg to about 10.0 μg/kg.

24. The method of claim 22, wherein said dose of N⁶-cyclopentyl-5′-(N-ethyl)carboxamidoadenosine is administered within from about 1 minute to about 30 minutes.

25. The method of claim 22, wherein said dose of N⁶-cyclopentyl-5′-(N-ethyl)carboxamidoadenosine is administered within about 15 minutes.

26. A dosing regime for treating an attack of atrial fibrillation or atrial flutter in a human, comprising an intravenous loading dose of an active agent which is N⁶-cyclopentyl-5′-(N-ethyl)carboxamidoadenosine or a pharmaceutically acceptable salt or ester thereof of from about 0.5 μg/kg to less than about 12.0 μg/kg, wherein said loading dose is administered to said human within from about 6 seconds to about 60 minutes, followed by an optional maintenance dose of said active agent as an intravenous infusion at a rate of from about 0.01 μg/kg/min to about 1.0 μg/kg/min.

27. The dosing regime of claim 26, wherein said loading dose is from about 1.25 μg/kg to less than about 12.0 μg/kg.

28. The dosing regime of claim 26, wherein said loading dose is administered within from about 30 seconds to about 60 minutes.

29. The dosing regime of claim 26, wherein said loading dose is administered within from about 1 minute to about 15 minutes.

30. The dosing regime of claim 26, wherein the maintenance dose is administered over from 1 hour to about 72 hours.

31. The dosing regime of claim 26, wherein the maintenance dose is administered up to 24 hours.

32. A method of achieving a therapeutic plasma concentration of an active agent which is N⁶-cyclopentyl-5′-(N-ethyl)carboxamidoadenosine or a pharmaceutically acceptable salt or ester thereof for treating atrial fibrillation or atrial flutter in a human in need of such treatment, comprising administering intravenously a loading dose of said active agent of from about more than 0.5 μg/kg to about 12.0 μg/kg, wherein said loading dose is administered to said human within from about 6 seconds to about 60 minutes, followed by an optional maintenance dose of said active agent as an intravenous infusion at a rate of from about 0.01 μg/kg/min to about 5 μg/kg/min.

33. The method of claim 32, wherein said loading dose is from about more than 1.25 μg/kg to about 12.0 μg/kg.

34. The method of claim 32, wherein said loading dose is administered within from about 30 seconds to about 60 minutes.

35. The method of claim 32, wherein said maintenance dose is administered at a rate of from 0.01 μg/kg/min to about 1.0 μg/kg/min.
